(19) United States
(12) Patent Application Publication
(10) Pub. No.: US 2026/0263351 A1
(43) Pub. Date: Sep. 10, 2026

(54) SPRAY COMPOSITION FOR POSTPARTUM PAIN RELIEF

(71) Applicant: TOREL HEALTHCARE, A DIVISION OF PEONY INVESTMENTS VMW LTD., Hamilton (CA)

(72) Inventor: VICTORIA WILSON, Dundas (CA)

(21) Appl. No.: 19/162,537

(22) PCT Filed: Mar. 6, 2024
(86) PCT No.: PCT/CA2024/050270

§ 371 Date: Sep. 5, 2025

Related U.S. Application Data
(60) Provisional application No. 63/488,884, filed on Mar. 7, 2023.

(51) Int. Cl. A61K 9/00; A61K 31/14; A61K 31/167; A61K 47/22; A61K 47/46

(52) U.S. Cl. CPC A61K 9/0014; A61K 31/14; A61K 31/167; A61K 47/22; A61K 47/46

(57) ABSTRACT

A topical spray composition for postpartum pain relief comprising lidocaine and benzalkonium chloride.

### FIELD OF THE INVENTION

The invention relates to a pharmaceutical spray composition for treatment of postpartum pain.

### BACKGROUND OF THE INVENTION

Most women experience some degree of pain or discomfort after childbirth. The pain is typically in the perineum, which may be bruised, or due to a tear or episiotomy. Pain is also typical at the incision site after a Cesarean birth.

Best practices for postpartum pain management are unfortunately quite primitive, and is a neglected area of clinical research (Fahey, J. Perinat Neonatal Nurs. 2017 Apr/Jun 31(2):126-136). These include administration of NSAIDs such as ibuprofen or a stronger prescription medication, exercise, breast feeding frequently, maintaining an empty bladder to alleviate pressure in the region, and the use of ice packs, or frozen menstrual pads (so called “padsicles”, which, combined with absorptive undergarments, significantly limit mobility). Inadequate pain relief can increase the risk of postpartum complications, increase the risk of chronic pain, impede mobility, interfere with maternal-newborn bonding and feeding, and create long term psychological associations of pain with childbirth. Further, since many postpartum women intend to breast feed, there is a risk or (at least) a perceived risk of transferring any pain medication such as opioids to the newborn through the breast milk, which creates a hesitancy to treat the pain aggressively using conventional systemic drugs. For example, codeine and tramadol are not recommended, because common genetic variants in CYP2D6, which metabolizes codeine and tramadol, can lead to toxic levels of active drug in breast milk (UNC Health Care Guidelines for Management of Postpartum Pain Following Discharge from Maternity Care, Mar. 16, 2018).

There is thus an unmet a need for localized postpartum pain relief.

Further, tears or lacerations in the area surrounding the vaginal opening, and areas surrounding stitches utilized to repair tears, Cesarean incision sites, or episiotomies, are prone to infection.

General topical pain relief sprays are known in the art. These include Dermoplast™, Solarcaine™, Bactine™, Earth Mama™ Herbal Perineal Spray, and Lansinoh™ Perineal Spray. Many of these provide aerosol sprays that soothe an area of interest, and most are indicated for “cuts, scrapes, minor burns, sunburn, or minor skin irritations”.

Antibacterial sprays are also known, though most are for treatment of hard surfaces. These include Microban™, Lysol™ disinfectant, 3M™ Cavilon™ No-Rinse Skin Cleanser, etc. However, most of these antibacterial sprays are designed for hard surfaces, hands, or other less sensitive areas, and there is a general reluctance to apply such sprays to sensitive regions such as the perineum or at a Cesarean incision site.

General topical pain relief sprays and/or antibacterial sprays are intended for easy to reach areas, and are therefore gravity-fed or aerosolized sprays. These are difficult to use in the perineum region, where the most straight-forward manner of self-administration of spray comprises turning the bottle upside down.

### BRIEF DESCRIPTION OF FIGURES

FIG. 1 is an illustration of a spray of the present invention.

### SUMMARY OF THE INVENTION

According to one aspect of the present invention is provided a liquid spray composition comprising: an effective amount of a local anesthetic, an effective amount of a biocide, and a carrier liquid.

In certain embodiments, the local anesthetic and/or the biocide are solubilized in the carrier liquid.

In certain embodiments, the local anesthetic and/or the biocide are in a suspension in the carrier liquid.

In certain embodiments, the local anesthetic is one or more of procaine, chloroprocaine, lidocaine, prilocaine, tetracaine, bupivacaine, cinchocaine, ropivacaine, benzocaine, chloroprocaine, cocaine, cyclomethycaine, dimethocaine, piperocaine, propoxycaine, procaine, proparacaine, tetracaine, articaine, bupivacaine, cinchocaine, etidocaine, levobupivacaine, lidocaine, mepivacaine, prioocaine, ropivacaine, trimecaine, saxitonin, neosaxitonin, tetrodotoxin, menthol, eugenol, or spilanthol.

In certain embodiments, the local anesthetic is a medium duration and medium potency local anesthetic.

In certain embodiments, the local anesthetic is one or more of lidocaine and prilocaine.

In certain embodiments, the biocide is an antimicrobial agent.

In certain embodiments, the antimicrobial agent is a germicide, an antibiotic, an antibacterial, an antiviral, an antifungal, an antiprotozoal, an antiseptic, or an antiparasite.

In certain embodiments, the antiseptic is one or more of a phenol, a diguanide, a quinolone, an alcohol, a peroxide, iodine, octenidine hydrochloride, benzalkonium chloride, cetylpyridinium chloride, or cetrimide.

In certain embodiments, the antiseptic is benzalkonium chloride.

According to certain embodiments, the local anesthetic is lidocaine and the biocide is benzalkonium chloride.

In certain embodiments, the liquid spray composition comprises 4-6% w/w lidocaine and 0.1-0.2% w/w benzalkonium chloride, for example, 5% w/w lidocaine and 0.13% w/w benzalkonium chloride.

In certain embodiments, the liquid spray composition further comprises one or more of aloe vera, vitamin E, and Witch Hazel.

According to a further aspect of the present invention is provided the liquid spray composition as hereindescribed, in a bag-on-valve aerosol spray container.

In certain embodiments, the bag-on-valve aerosol spray container allows for 360° rotation of the spray when utilized.

According to a further aspect of the present invention is provided a method of treating post-partum pain from episiotomy or natural tearing during childbirth, comprising administration of an effective amount of the liquid spray composition as hereindescribed to a human female in need thereof.

According to a further aspect of the present invention is provided the liquid spray composition as hereindescribed, use in treating postpartum pain from episiotomy or natural tearing during childbirth in a human female in need thereof.

According to a further aspect of the present invention is provided the use of the liquid spray composition as hereindescribed in treatment of post-partum pain from episiotomy or natural tearing during childbirth in a human female in need thereof.

### DESCRIPTION

A spray for use in postpartum pain relief, comprising a local anesthetic in combination with a biocide, such as an antibacterial or antimicrobial agent. The local anesthetic and biocide are dissolved or suspended in a spray liquid suitable for localized topical administration.

The liquid spray composition contains a local anesthetic. The local anesthetic can be any known local anesthetic suitable for localized, local pain relief, and may be for example procaine, chloroprocaine, lidocaine, prilocaine, tetracaine, bupivacaine, cinchocaine, ropivacaine, benzocaine, chloroprocaine, cocaine, cyclomethycaine, dimethocaine, piperocaine, propoxycaine, procaine, proparacaine, tetracaine, articaine, bupivacaine, cinchocaine, etidocaine, levobupivacaine, lidocaine, mepivacaine, prioocaine, ropivacaine, trimecaine, saxitonin, neosaxitonin, tetrodotoxin, menthol, eugenol, or spilanthol, or any suitable combination thereof.

In a preferred embodiment, the local anesthetic is a medium duration and medium potency local anesthetic, such as lidocaine and/or prilocaine. The local anesthetic may be in a concentration already known to be effective for localized, local pain relief, or it may be in a concentration tuned for use in postpartum pain relief. In preferred exemplifications, the local anesthetic is provided at the maximum allowable and approved concentration for use as a topical spray formulation. For example, the local anesthetic may be a 4-6% w/w, such as a 5% w/w lidocaine solution, which is a concentration approved or approvable for sale without a prescription in the United States and in other countries. In other embodiments, a stronger concentration of the local anesthetic may be used, for over the counter or prescription use, or for use in hospital settings under the supervision of a doctor.

The liquid spray composition also includes a biocide, for example, an antimicrobial agent. The antimicrobial agent may be any agent known to be useful as a topical antimicrobial, and may be a germicide, an antibiotic, an antibacterial, an antiviral, an antifungal, an antiprotozoal, an antiseptic, and/or an antiparasite agent. The antiseptic may be a phenol, a diguanide, a quinolone, an alcohol, a peroxide, iodine, octenidine hydrochloride, benzalkonium chloride, cetylpyridinium chloride, cetrimide, or any combination thereof. For example, the biocide may be the antiseptic, benzalkonium chloride.

The biocide may be in a concentration already known to be effective in a topical spray form, or it may be in a concentration tuned for use for postpartum injuries. In preferred exemplifications, the biocide is provided at the maximum allowable and approved concentration for use as a topical spray formulation. For example, the biocide may be a 0.1-0.2% w/w, such as a 0.14% w/w benzalkonium chloride solution, which is a concentration approved or approvable for sale without a prescription in the United States and in other countries. In other embodiments, a stronger concentration of the biocide may be used, for over the counter or prescription use, or for use in hospital settings under the supervision of a doctor.

In a preferred embodiment, the topical spray is a suspension or solution (preferably a solution) comprising 0.14% w/w benzalkonium chloride and 5% w/w lidocaine.

The topical spray may be a suspension or solution in any suitable solvent or suspension agent. For example, the solution or suspension may be an ethanol solution, or an ethanol/water solution.

The topical spray may include other ingredients such as non-medicinal ingredients known or believed to aid in wound repair or treatment, or to soothe skin, such as aloe vera or Aloe Barbadensis leaf juice, Witch Hazel (Hamamelis virginana), Vitamin E, Castor oil, and the like. These other ingredients may be in varying concentrations, for example, 0.1% w/w to 5% w/w of aloe vera, Witch Hazel, and/or Vitamin E.

The topical spray may also contain emulsifying agents, suspension agents, carriers, preservatives, scent agents, and other ingredients typical for a medicinal topical spray.

In a preferred embodiment, the topical spray is contained within a spray bottle that provides a relatively evenly dispersed spray when activated. The spray bottle may be a conventional spray bottle, or may be a spray bottle which allows for spraying when the bottle is inverted, allowing for a 360° rotation of the spray bottle to facilitate self-administration to the perineum area. This is an important benefit, since with a traditional spray bottle it may be difficult for a person to configure their handling of the spray bottle to effectively self-administer the spray to this region of the body. For example, a bag-on-valve aerosol spray may be utilized, which allows for 360° rotation of the spray bottle (i.e. the bottle may be used upside down). Bag-on-valve aerosol products have the added benefit of providing a more even and finer spray molecular weight, which allows for easier absorbtion of the spray into the skin. Bag on valve technology has several advantages ideal for a topical spray formulation, since it provides high shelf life, an even and finer mist, the ability to spray in any direction, and the added benefit, for a pharmaceutical product, that the aerosol gas and the topical spray formulation are separate, which means the aerosol gas does not act to oxidize or otherwise degrade the spray formulation.

By using a bag-on-valve aerosol spray, one also avoids the undesirable greasy residue, that sticks to the skin, that is found in prior art sprays such as Dermoplast™ or Solarcaine™. The undesirable oily feeling is caused by the propellant. A bag-on-valve system allows for use of a water-soluble solution, which does not have said oily or greasy residue sensation when applied.

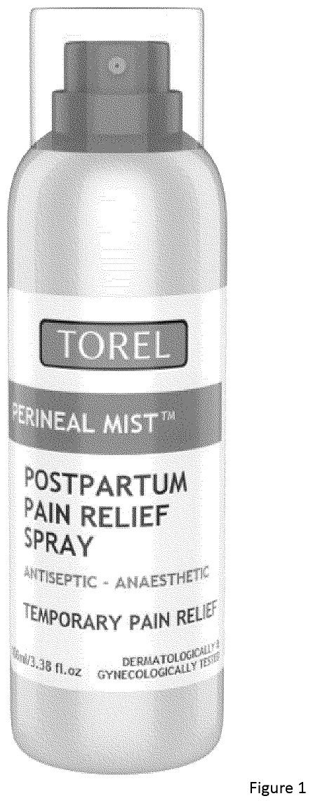

FIG. 1 shows a bag-on-valve aerosol spray bottle containing a medicinal topical spray of the present invention.

The topical spray composition provides excellent pain relief and antibacterial effect to aid in healing tears or incisions postpartum.

1. A liquid spray composition comprising: an effective amount of a local anesthetic, an effective amount of a biocide, and a carrier liquid.

2. The liquid spray composition of claim 1 wherein the local anesthetic and/or the biocide are solubilized in the carrier liquid.

3. The liquid spray composition of claim 1 wherein the local anesthetic and/or the biocide are in a suspension in the carrier liquid.

4. The liquid spray composition of claim 1 wherein the local anesthetic is one or more of procaine, chloroprocaine, lidocaine, prilocaine, tetracaine, bupivacaine, cinchocaine, ropivacaine, benzocaine, chloroprocaine, cocaine, cyclomethycaine, dimethocaine, piperocaine, propoxycaine, procaine, proparacaine, tetracaine, articaine, bupivacaine, cinchocaine, etidocaine, levobupivacaine, lidocaine, mepivacaine, prioocaine, ropivacaine, trimecaine, saxitonin, neosaxitonin, tetrodotoxin, menthol, eugenol, or spilanthol.

5. The liquid spray composition of claim 1 wherein the local anesthetic is a medium duration and medium potency local anesthetic.

6. The liquid spray composition of claim 5 wherein the local anesthetic is one or more of lidocaine and prilocaine.

7. The liquid spray composition of claim 1 wherein the biocide is an antimicrobial agent.

8. The liquid spray composition of claim 7 wherein the antimicrobial agent is a germicide, an antibiotic, an antibacterial, an antiviral, an antifungal, an antiprotozoal, an antiseptic, or an antiparasite.

9. The liquid spray composition of claim 8 wherein the antiseptic is one or more of a phenol, a diguanide, a quinolone, an alcohol, a peroxide, iodine, octenidine hydrochloride, benzalkonium chloride, cetylpyridinium chloride, or cetrimide.

10. The liquid spray composition of claim 9 wherein the antiseptic is benzalkonium chloride.

11. The liquid spray composition of claim 1 wherein the local anesthetic is lidocaine and the biocide is benzalkonium chloride.

12. The liquid spray composition of claim 11 comprising 4-6% w/w lidocaine and 0.1-0.2% w/w benzalkonium chloride.

13. The liquid spray composition of claim 12 comprising 5% w/w lidocaine and 0.13% w/w benzalkonium chloride.

14. The liquid spray composition of claim 1 further comprising one or more of aloe vera, vitamin E, and Witch Hazel.

15. The liquid spray composition of claim 1, in a bag-on-valve aerosol spray container.

16. The liquid spray composition of claim 15 wherein the bag-on-valve aerosol spray container allows for 360° rotation of the spray when utilized.

17. Method of treating post-partum pain from episiotomy or natural tearing during childbirth, comprising administration of an effective amount of the liquid spray composition of claim 1.

18. The liquid spray composition of claim 1 for use in treating postpartum pain from episiotomy or natural tearing during childbirth in a human female in need thereof.

19. Use of the liquid spray composition of claim 1 in treatment of post-partum pain from episiotomy or natural tearing during childbirth in a human female in need thereof.
